(19) United States
(12) Patent Application Publication
(10) Pub. No.: US 2025/0295500 A1
(43) Pub. Date: Sep. 25, 2025

(54) SYSTEM FOR EXTREMITIES AND RELATED METHODS

(71) Applicant: Forma Medical, Inc., Camp Hill, PA (US)

(72) Inventors: James A. Gault, Lincoln, RI (US); Andrew Charles Davison, Hummelstown, PA (US)

(73) Assignee: Forma Medical, Inc., Camp Hill, PA (US)

(21) Appl. No.: 19/084,023

(22) Filed: Mar. 19, 2025

Related U.S. Application Data
(60) Provisional application No. 63/567,081, filed on Mar. 19, 2024.

(51) Int. Cl. A61F 2/46; A61B 17/00; A61B 17/34; A61B 17/88; A61B 17/90; A61B 90/00; A61F 2/42

(52) U.S. Cl. CPC A61F 2/4606; A61B 17/3468; A61B 17/3472; A61B 17/8897; A61B 17/90; A61F 2/4225; A61B 2017/0092; A61B 2090/3966; A61F 2002/4677; A61F 2002/4687

(57) ABSTRACT

A system configured to guide one or more wires or fixation elements toward one or more bone segments of extremity bones is provided.

### CROSS-REFERENCE TO RELATED APPLICATIONS

The present application claims the benefit of and priority to U.S. Provisional Application No. 63/670,414, filed Jul. 12, 2024, the entire contents of which are incorporated by reference into this application.

### TECHNICAL FIELD

The present disclosure relates generally to a system for extremity bones and related methods, in particular, to an aiming guide system.

### BACKGROUND

A common procedure for handling healing of broken bones and addressing deformities such as hammertoe is the use of bone fixation implants for fusing one or more adjacent bones. Conventional bone fixation implants utilize generic screws and wires that create a rigidly fused joint with very limited adjustability intraoperatively. Some implants offer some limited degree of flexibility and/or adjustment when used under very specific circumstances that require highly technical surgical procedures. Such existing bone fixation implants often require multiple components with many intricate mating features requiring customization depending on the type of bone, patient, or desired location of the implant in the body of a patient. This results in increased costs, less desirable healing outcomes, and multiple procedures to achieve a desired outcome.

### SUMMARY

An embodiment of the present disclosure includes a system for an extremity bone. The system includes a surgical instrument having a first guide component and a proximal guide component aligned along a guide axis. The first guide component and the second guide component can define a gap therebetween sized to permit a portion of the extremity bone to fit therein. The first guide component has a first component body defining a first guide channel that extends along the guide axis and that is sized and shaped to receive a wire. The first guide body is radiopaque. The second guide component has a second component body defining a second guide channel that extends along the guide axis, and an elongated slot that opens to the second guide channel. The second component body is radiolucent. The first guide component is configured to engage the extremity bone.

In the system, the first component body includes a base and first tip that extends from the base, where the first component body defines a first opening and a second opening opposite the first opening, such that, a first end of the wire is insertable into the second opening. The first component body defines a slot that opens into the first guide channel such that a wire is removable in a direction that is transverse with respect to the guide axis. The second component body may include a connecting leg that couples the first guide component to the second guide component and a gripping member that extends from the leg. The second component body also has a first end, a second end opposite the first end along the guide axis, and the elongated slot extends in a direction aligned with the guide axis. The first guide channel has a first cross-sectional dimension that is perpendicular to the guide axis, and the second guide channel has a second cross-sectional dimension that is perpendicular to the guide axis, where the second cross-sectional dimension is greater than the first cross-sectional dimension. The system may include a trocar having a proximal head, a shaft that extends from the proximal head in a first direction, a wire channel that extends from the proximal head through the shaft to a first end of the trocar.

The shaft has a first portion configured to engage bone and a second portion that is sized and shaped to slidingly fit within the second guide channel but not fit in the first guide channel.

In the system, the first guide channel has a first cross-sectional dimension that is perpendicular to the guide axis, and the second guide channel has a second cross-sectional dimension that is perpendicular to the guide axis, where the second cross-sectional dimension is greater than the first cross-sectional dimension, such that, the shaft of the trocar is insertable into the second guide channel but is not insertable into the first guide channel. The shaft of the trocar extends along a shaft axis and a first portion of the shaft has a first cross-sectional dimension that is perpendicular to the shaft axis, and the second portion of the shaft has a second cross-sectional dimension that is perpendicular to the shaft axis, with the second cross-sectional dimension being greater than the first cross-sectional dimension. The wire channel has a first opening at the first end and a second opening at the second head. And the proximal head defines a chamfer that extends to the second opening to facilitate insertion of the wire. The second portion of the shaft includes cutting flutes.

In another embodiment, the system includes a surgical instrument having a first guide component and a second guide component aligned along a guide axis. The first guide component having a first component body defining a first guide channel that extends along the guide axis and that is sized and shaped to receive a wire. The first guide body is formed from a radiopaque metal. The surgical instrument also includes the second guide component having a second component body. The second component body defining a second guide channel that extends along the guide axis, and an elongated slot that opens to the second guide channel. The second component body is formed from a radiolucent polymer. The system also includes a trocar having a proximal head, a shaft that extends from the proximal head in a first direction, and a wire channel that extends from the proximal head through the shaft to a first end of the trocar. The shaft has a first portion configured to engage bone and a second portion that is sized and shaped to slidingly fit within the second guide channel but not to engage the first guide channel.

In the system, the first component body includes a base and first tip that extends from the base. The first component body has a first opening and a second opening opposite the first opening, such that, a first end of the wire is insertable into the second opening. The first component body defines a slot that opens into the first guide channel such that a wire is removable in a direction that is transverse with respect to the guide axis. The second component body may include a connecting leg that couples the first guide component to the second guide component and a gripping member that extends from the leg. The second component body has a first end, a second end opposite the first end along the guide axis with the elongated slot extends in a direction aligned with the guide axis. The first guide channel has a first cross-sectional dimension that is perpendicular to the guide axis and the second guide channel has a second cross-sectional dimension that is perpendicular to the guide axis with the second cross-sectional dimension being greater than the first cross-sectional dimension, such that, the shaft of the trocar is insertable into the second guide channel but is not insertable into the first guide channel. The shaft of the trocar extends along a shaft axis, where the first portion of the shaft has a first cross-sectional dimension that is perpendicular to the shaft axis, and the second portion of the shaft has a second cross-sectional dimension that is perpendicular to the shaft axis, where the second cross-sectional dimension is less than the first cross-sectional dimension.

Another embodiment of the present disclosures includes a method. The method includes forming a pilot hole in a first bone and inserting a first tip of a guide instrument into the pilot hole in a first bone such that a first guide component of the guide instrument is engaged with the first bone and a second guide component of the guide instrument is aligned with a second bone that is first to the first bone. The method also includes inserting a trocar over the wire and into the first guide channel and the wire channel until a portion of a shaft of the trocar engages the first guide component. The method includes rotating the trocar to engage the second bone. The method includes inserting a wire through a wire channel of the second guide component and a first guide channel of the first guide component until a first end of the wire engages the first bone. The method also includes removing the trocar from the wire, and the wire channel of the second guide component and the distal guide channel of the distal guide component. The method includes lifting the guide instrument in a dorsal direction so that the wire exits a slot of the first guide component and a slot of the second guide component.

### BRIEF DESCRIPTION OF THE DRAWINGS

The foregoing summary, as well as the following detailed description of exemplary embodiments of the present application, are better understood when read in conjunction with the appended drawings. For the purposes of illustrating the present application, shown in the drawings are exemplary embodiments of the disclosure. It should be understood, however, that the application is not limited to the precise arrangements and instrumentalities shown. In the drawings:

FIG. 1 is a top view of an anatomical foot;

FIG. 2 is a side view of a system according to an embodiment of the present disclosure;

FIG. 3 is a side view of a guide instrument of the system shown in FIG. 2;

FIG. 4 is a bottom of the guide instrument shown in FIG. 2;

FIG. 5 is a side view of a trocar of the system shown in FIG. 2;

FIG. 6 is a perspective view of the trocar shown in FIG. 5; and

FIGS. 7-14 illustrate a method for guiding a device toward a bone according to an embodiment of the present disclosure.

### DETAILED DESCRIPTION OF ILLUSTRATIVE EMBODIMENTS

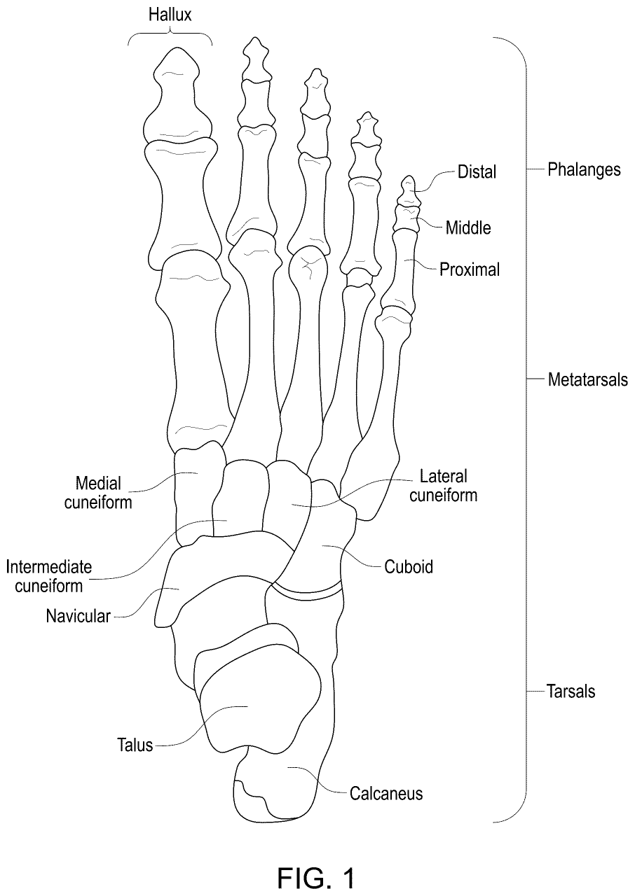

Systems and fixation devices as described are configured for aid in the fixation of two or more bones or bone segments, typically in extremity bones, such as the foot. As shown in FIG. 1, the skeletal anatomy of a foot includes tarsals, metatarsals, and phalanges. The foot bone structure is further typically divided into three regions: the hindfoot, midfoot, and forefoot. The tarsal bones are seven bones in the hindfoot and midfoot and include the calcaneus, talus, cuboid, navicular, and three cuneiforms. The metatarsal bones are five bones in the forefoot that connect the tarsals to the phalanges. The phalanges in the forefoot that form the toes. The systems as described herein are configured for fixation of toe and/or for hammertoe correction, for example. In other examples, the systems and methods described herein are configured for interphalangeal joint fixation. For example, the systems may be used for, or to aid in fixation of metatarsals, proximal phalanges, middle phalanges, or distal phalanges. While the embodiments described are configured for interphalangeal joint fixation, it is possible that the described embodiments could be configured for fixation of phalanges, metatarsals, cuneiform, or cuboid bones in the foot. In other embodiments, systems may be used for fixation of bone segments of phalanges, metatarsals, or other bones in the hand.

The present disclosure s terms like distal and proximal to indicate reference to the devices and system components being described. The term “distal” shall mean away from the center of a body (or device). The term “proximal” shall mean closer towards the center of a body (or device) and/or away from the “distal” end. Thus, proximal typically means closer to the user or surgeon and the distal refers to the further away from the user or surgeon, relative to the device, instrument or anchor, etc. “Distal” and “proximal” and may be interchangeably used with “first” and “second,” respectively, as needed based on context. Such directional terms used in conjunction with the description of the drawings should not be construed to limit the scope of the present disclosure in any manner not explicitly stated here.

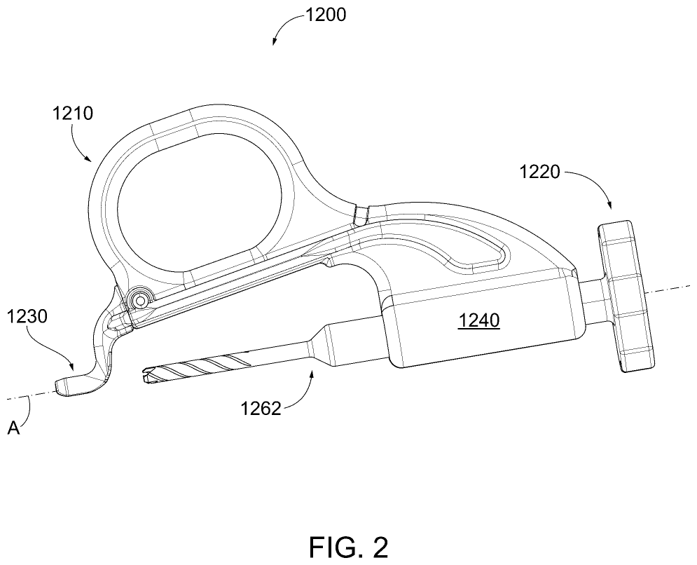

As shown in FIG. 2, a system 1200 may be used to guide various devices, e.g. instruments, wires, anchors, and other fixation elements toward a target bone. The system 1200 includes a surgical instrument 1210 and a trocar 1220. Additional components, such as wires, fixation elements, drills, cannulas and the like may also be included in system 1200. For instance, the system may include a bone anchor for inserting through the first and second bones along a wire.

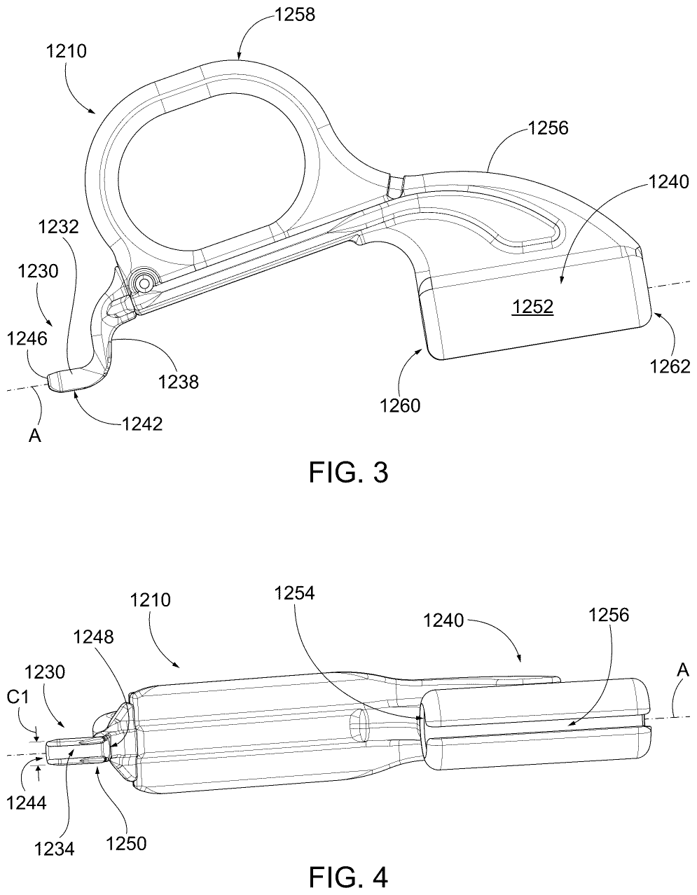

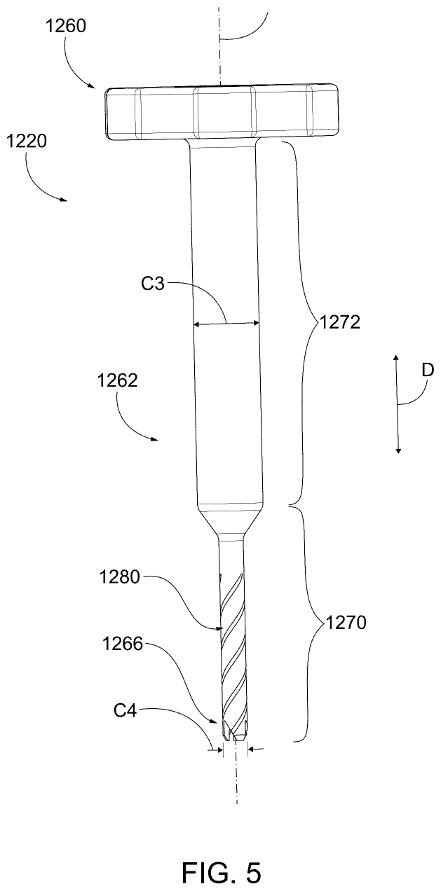

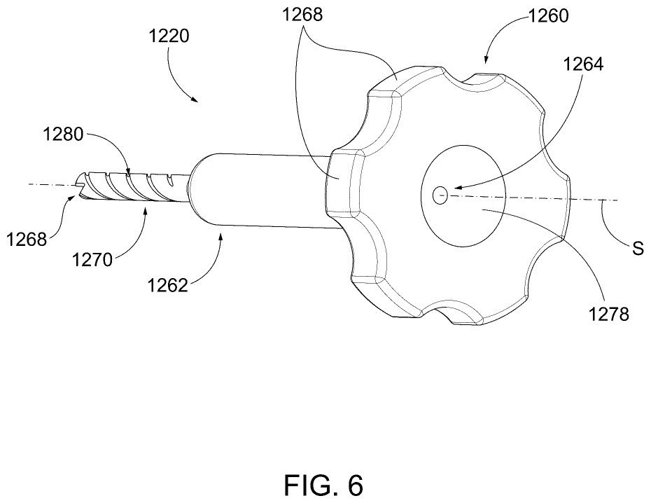

Referring to FIGS. 2-4, the instrument 1210 has a distal guide component 1230 and a proximal guide component 1240 aligned along a guide axis A. The distal guide component 1230 may be referred to a first guide component and the proximal guide component 1240 may be referred to a second guide component as needed. The distal guide component 1230 and the proximal guide component 1240 are arranged on the axis A to define a gap therebetween sized to permit a portion of the extremity bone to fit therein. For example, the distal guide component 1230 can engage a first bone segment, and the instrument can be arranged, with the distal guide component engaging the first bone segment, so the proximal guide component 1240 engages a second bone segment. The gap also provides for space for the trocar 1220 to engage the bone segments when the trocar 1220 is inserted through the proximal guide component 1240 as described further below.

The distal guide component 1230 has a distal component body 1232 defining a distal guide channel 1234. The distal guide channel 1234, in turn, extends along the guide axis A. The distal guide channel 1234 is sized and shaped to receive a wire. For instance, the distal guide channel 1234 has a first cross-sectional dimension C1 that is perpendicular to the guide axis A.

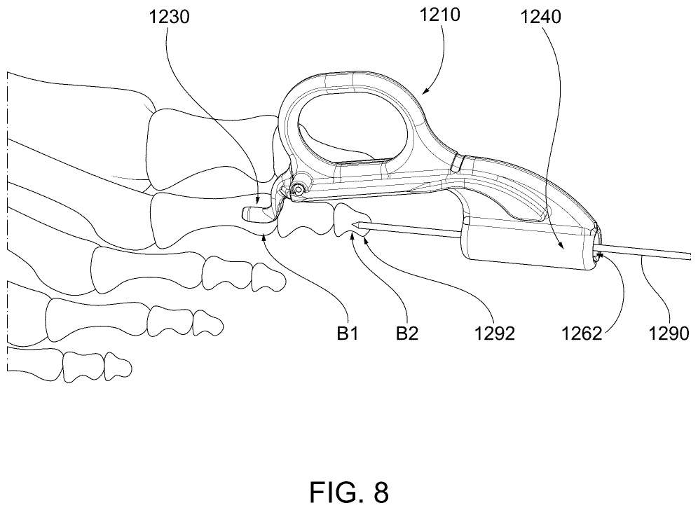

The distal component body 1232 also includes a base 1238 and a curved distal tip 1242 that extends from the base 1238. The distal component body 1232 further defines a distal opening 1244, a distal-most end 1246 of the curved distal tip 1242 and a proximal opening 1248 opposite the distal opening 1244. Configured this way, a distal end 1292 of the wire 1290 (FIG. 8) is insertable into the proximal opening 1248. The distal component body 1232 further defines a slot 1250 that opens into the distal guide channel 1234 such that a wire is removable in a direction that is transverse with respect to the guide axis A. The distal component body 1232 may radiopaque. For example, the distal component body 1232 may be metal, and particularly, a radiopaque metal or a polymer with radiopaque elements, such as additives and the like, incorporate therein. Thus, at least the distal tip 1242 is configured for visualization using typical radiographic techniques.

The proximal guide component 1240 has a proximal component body 1252. The proximal component body 1252 defines a proximal guide channel 1254 that extends along the guide axis A and an elongated slot 1256 that opens to the proximal guide channel 1254. The proximal component body 1252 may include a connecting leg 1256 that couples the distal guide component 1230 to the proximal guide component 1240, and a gripping member 1258 that extends from the leg 1256. The proximal component body 1252 further has a distal end 1260 and a proximal end 1262 opposite the distal along the guide axis A. The elongated slot 1256 extends in a direction aligned with the guide axis A. The proximal guide channel 1254 has a second cross-sectional dimension C2 (not shown) that is perpendicular to the guide axis A. However, the second cross-sectional dimension C2 is greater than the first cross-sectional dimension C1, such that, the shaft 1262 of the trocar 1220 is insertable into the proximal guide channel 1254 but is not insertable into the distal guide channel 1234. The proximal component body 1252 is formed from a polymer. In one example, the polymer is a radiolucent polymer that is not typically capable of visualization by X-rays are similar imaging systems.

The system 1200 also includes a trocar 1220 configured to engage the instrument along the guide axis A. The trocar has a proximal head 1260, a shaft 1262 that extends from the proximal head 1260 in a distal direction D along a shaft axis S, and a wire channel 1264 that extends from the proximal head 1262 through the shaft 1262 to a distal end 1266 of the trocar 1220. The proximal head 1260 includes one or more gripping members 1268. The shaft 1262 has a first portion 1272 that is sized and shaped to slidingly fit within the proximal guide channel 1254, and a second portion 1270 with the distal end 1266 configured to engage bone. The wire channel 1264 has a distal opening at the distal end 1266, and a proximal opening at the proximal head 1260. The proximal head 1260 defines a chamfer 1278 that extends into or toward the proximal opening to facilitate insertion of the wire. More specifically, the first portion 1272 of the shaft 1262 has a first cross-sectional dimension C3 that is perpendicular to the shaft axis S, the second portion 1270 of the shaft 1262 has a second cross-sectional dimension C4 that is perpendicular to the shaft axis S and the second cross-sectional dimension C4 is less than the first cross-sectional dimension C1. The first portion 1272 of the shaft 1262 has a smooth outer surface to slidingly fit in the proximal guide channel 1254. In the example shown, the first portion 1272 of the shaft 1262 has length that is greater than a length of the second portion 1270 of the shaft 1262. The first portion 1270 of the shaft 1262 defines a distal end 1266 of the trocar. The first portion 1270 of the shaft 1262 also includes cutting flutes 1280. The trocar 1220 may be radiopaque. For instance, the trocar 1220 may be radiopaque. For instance the trocar may be made of a radiopaque metal and or a polymer with radiopaque elements or additives incorporated therein. Thus, at least the trocar 1220 is configured for visualization using typical radiographic techniques along with the distal tip and a wire as needed.

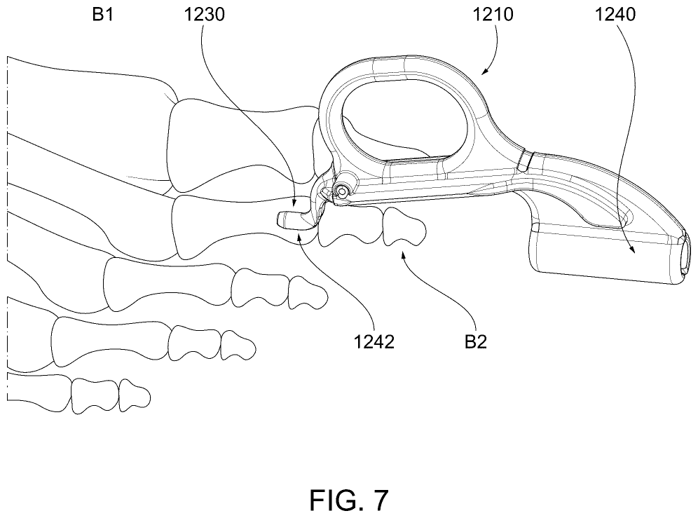

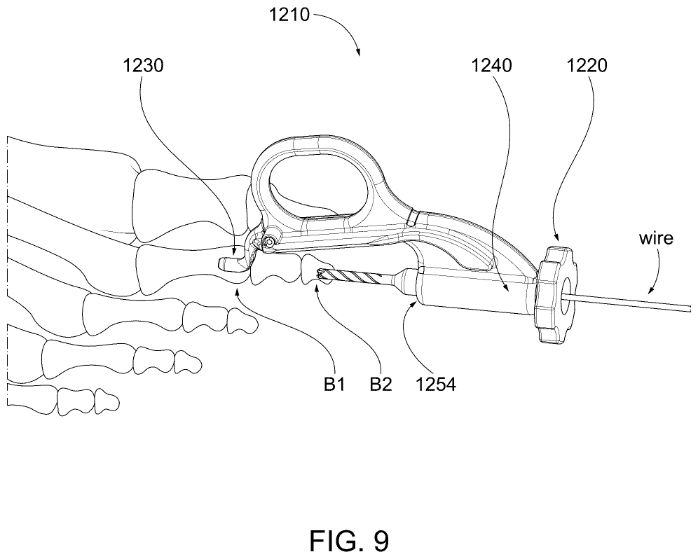

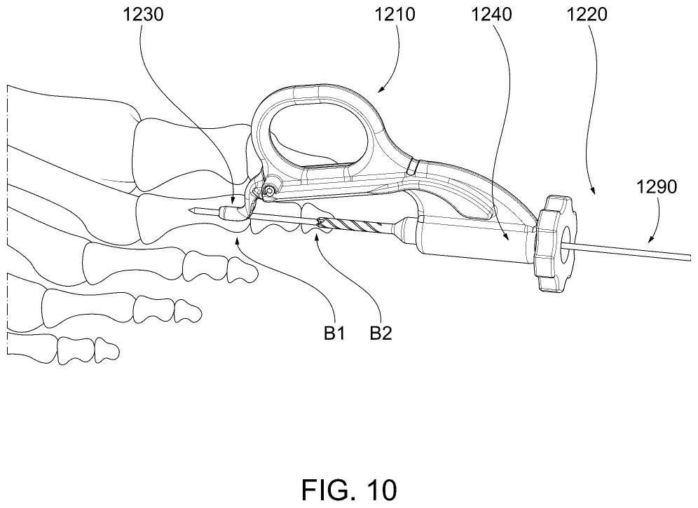

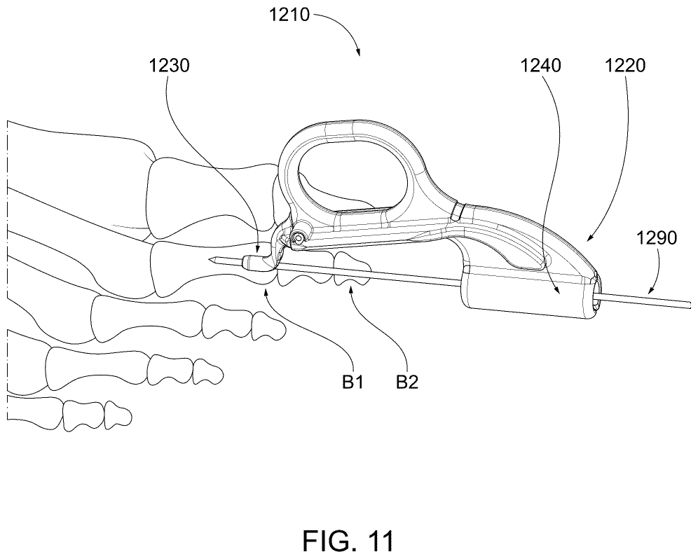

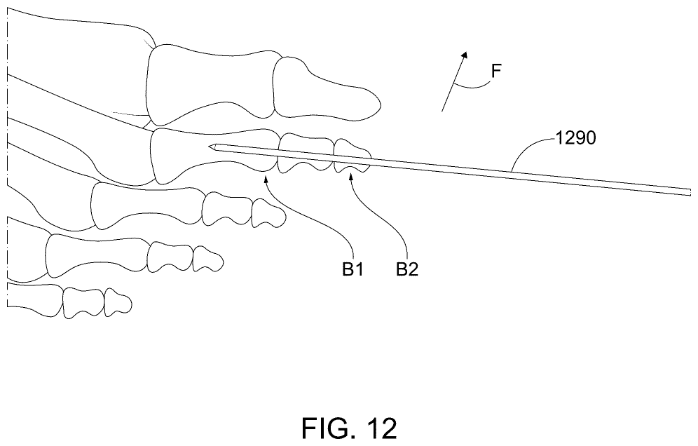

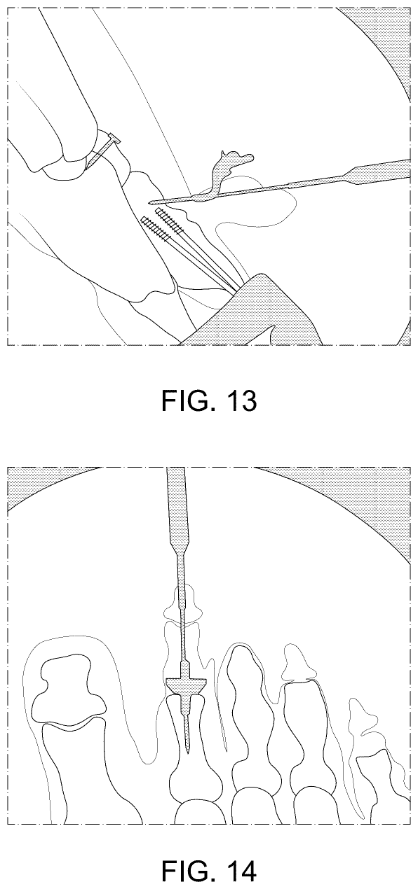

FIGS. 7-14 illustrate a method for using a system 1200. The method may include initially forming a pilot hole in a first bone B1 (e.g. a proximal phalanx). In FIG. 7, the method also includes inserting a distal tip 1242 of a guide instrument 1210 into the pilot hole, such that a distal guide component 1230 of the guide instrument is engaged with the first bone B1 and a proximal guide component 1240 of the guide instrument is aligned with a second bone B2 (e.g. distal phalanx) that is distal to the first bone B1. As shown in FIG. 9, the method may include inserting a trocar 1220 over the wire 1290 and into the guide channel 1254. The method also includes rotating the trocar 1220 to engage the second bone B2. Then, the user or surgeon inserts a wire 1290 through the wire channel in the trocar 1220 and wire channel 1234 of the distal guide component 1230 (via the proximal guide body) until a distal end 1292 of the wire 1290 engages the second bone B2 and enters the first bone B1, which may be about 5 mm into the proximal phalanx. as shown in FIG. 10. As shown in FIG. 11, the method also includes removing the trocar 1220 from the wire 1290 and the channel 1234 of the distal guide component 1230 and channel of the proximal guide component. The method also includes lifting the guide instrument 1210 in a dorsal direction F so that the wire exits a slot of the distal guide component and the slot of the proximal guide component and surgical instrument is removed. As shown in FIGS. 13 and 14, which are schematics of X-rays images showing the radiopaque distal tip of the guide, the trocar, and the wire.

Wherever possible, the same or like reference numbers are used throughout the drawings and description to refer to the same or like features. It should be noted that the drawings are in a simplified schematic form and are not drawn to precise scale. Certain terminology used in the description is for convenience only and is not limiting. Directional terms such as top, bottom, left, right, above, below and diagonal, are used with respect to the accompanying drawings. Additionally, the term “a”, as used in the specification, means “at least one.” The terminology includes the words above specifically mentioned, derivatives thereof, and words of similar import.

Furthermore, the described features, advantages and characteristics of exemplary embodiments may be combined in any suitable manner in one or more embodiments. One skilled in the art will recognize, in light of the description herein, that the exemplary embodiments can be practiced without one or more of the specific features or advantages of a particular embodiment. In other instances, additional features and advantages may be recognized in certain embodiments that may not be present in all embodiments of the present disclosure.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to a sequential series of reference signs and progression of the blocks in the figures, the method can be implemented in an order as desired.

1. A system for an extremity bone, comprising:
a surgical instrument having a first guide component and a second guide component aligned along a guide axis and separated by gap,
the first guide component having a first component body defining a first guide channel that extends along the guide axis and that is sized and shaped to receive a wire, and the first guide body is radiopaque, wherein the first guide component is configured to engage the extremity bone.
the second guide component having a second component body, the second component body defining a second guide channel that extends along the guide axis, and an elongated slot that opens to the second guide channel, and the second component body is radiolucent.

2. The system according to claim 1, wherein the first component body includes a base and a first tip that extends from the base, wherein the first component body has a first opening and a second opening opposite the first opening, such that, a first end of a wire is insertable into the second opening.

3. The system according to claim 1, wherein the first component body defines a slot that opens into the first guide channel such that a wire is removable in a direction that is transverse with respect to the guide axis.

4. The system according to claim 1, wherein the second component body comprises:
a connecting leg that couples the first guide component to the second guide component; and
a gripping member that extends from the leg.

5. The system according to claim 1, wherein the second component body has a first end, a second end opposite the first end along the guide axis, wherein the elongated slot extends in a direction aligned with the guide axis from the first end toward the second end.

6. The system according to claim 1, wherein the first guide channel has a first cross-sectional dimension that is perpendicular to the guide axis, and the second guide channel has a second cross-sectional dimension that is perpendicular to the guide axis, wherein the second cross-sectional dimension is greater than the first cross-sectional dimension.

7. The system according to claim 1, further comprising a trocar having a proximal head, a shaft that extends from the proximal head in a first direction, a wire channel that extends from the proximal head through the shaft to a first end of the trocar, the shaft having a first portion configured to engage bone, and a second portion that is sized and shaped to slidingly fit within the second guide channel but not to fit within the first guide channel.

8. The system according to claim 7, wherein the trocar is radiopaque.

9. The system according to claim 7, wherein the first guide channel has a first cross-sectional dimension that is perpendicular to the guide axis, and the second guide channel has a second cross-sectional dimension that is perpendicular to the guide axis, wherein the second cross-sectional dimension is greater than the first cross-sectional dimension, such that, the shaft of the trocar is insertable into the second guide channel but is not insertable into the first guide channel.

10. The system according to claim 7, wherein the shaft of the trocar extends along a shaft axis, wherein the first portion of the shaft has a first cross-sectional dimension that is perpendicular to the shaft axis, and the second portion of the shaft has a second cross-sectional dimension that is perpendicular to the shaft axis, wherein the first cross-sectional dimension is less than the second cross-sectional dimension.

11. The system according to claim 7, wherein the wire channel has a first opening at the first end and a second opening at the proximal head, wherein the proximal head defines a chamfer that extends to the second opening to facilitate insertion of the wire.

12. The system according to claim 7, wherein the proximal head includes one or more gripping members.

13. The system according to claim 7, wherein the second portion of the shaft includes cutting flutes.

14. A system, comprising:
a surgical instrument having a first guide component and a second guide component aligned along a guide axis and separated by a gap,
the first guide component having a first component body defining a first guide channel that extends along the guide axis and that is sized and shaped to receive a wire, wherein the first guide body is radiopaque,
the second guide component having a second component body, the second component body defining a second guide channel that extends along the guide axis, and an elongated slot that opens to the second guide channel, wherein the second component body is radiolucent; and
a trocar having a proximal head, a shaft that extends from the proximal head in a first direction, a wire channel that extends from the proximal head through the shaft to a first end of the trocar, the shaft having a first portion configured to engage bone and a second portion proximal to the first portion that is sized and shaped to slidingly fit within the second guide channel but not to fit within the first guide channel.

15. The system according to claim 14, wherein the first component body includes a base and first tip that extends from the base, wherein the first component body defines a first opening and a second opening opposite the first opening, such that, a first end of the wire is insertable into the second opening.

16. The system according to claim 14, wherein the first component body defines a slot that opens into the first guide channel such that a wire is removable in a direction that is transverse with respect to the guide axis.

17. The system according to claim 14, wherein the second component body comprises:
a connecting leg that couples the first guide component to the second guide component; and
a gripping member that extends from the leg.

18. The system according to claim 14, wherein the second component body has a first end, a second end opposite the first along the guide axis, and wherein the elongated slot extends in a direction aligned with the guide axis.

19. The system according to claim 14, wherein the first guide channel has a first cross-sectional dimension that is perpendicular to the guide axis, and the second guide channel has a second cross-sectional dimension that is perpendicular to the guide axis, wherein the second cross-sectional dimension is greater than the first cross-sectional dimension, such that, the shaft of the trocar is insertable into the second guide channel but is not insertable into the first guide channel.

20. The system according to claim 14, wherein the shaft of the trocar extends along a shaft axis, wherein the first portion of the shaft has a first cross-sectional dimension that is perpendicular to the shaft axis, and the second portion of the shaft has a second cross-sectional dimension that is perpendicular to the shaft axis, wherein the second cross-sectional dimension is less than the first cross-sectional dimension.

21. A method, comprising:
forming a pilot hole in a first bone;
inserting a first tip of a guide instrument into a pilot hole in a first bone such that a first guide component of the guide instrument is engaged with the first bone and a second guide component of the guide instrument is aligned with a second bone that spaced from the first bone;
inserting a trocar into the first guide channel until a portion of a shaft of the trocar engages first guide component;
rotating the trocar to engage the second bone;
inserting a wire through a wire channel of the second guide component and a first guide channel of the first guide component until a first end of the wire engages the first bone;
removing the trocar from the wire, the wire channel of the second guide component, and the first guide channel of the first guide component;
lifting the guide instrument in a dorsal direction so that the wire exits a slot of the first guide component and a slot of the second guide component.

22. The method according to claim 21, further comprising inserting a bone anchor over the wire into engagement with the second bone and the first bone.

23. The method according to claim 22, wherein the first tip of a guide instrument is radiopaque, and the trocar is radiopaque while the second guide component is not radiopaque.
